(19) United States
(12) Patent Application Publication
(10) Pub. No.: US 2011/0300628 A1
(43) Pub. Date: Dec. 8, 2011

(54) ENHANCEMENT OF EPIDERMAL CELL GROWTH BY NON-PROTEIN GROWTH FACTORS

(76) Inventor: JOHN JACOB WILLE, JR., Chesterfield, NJ (US)

(21) Appl. No.: 13/150,421

(22) Filed: Jun. 1, 2011

Related U.S. Application Data
(60) Provisional application No. 61/351,312, filed on Jun. 4, 2010.

(51) Int. Cl. C12N 5/071

(52) U.S. Cl. 435/371

(57) ABSTRACT

The serum-free culture of normal human epithelial stem cells is of paramount importance for the in vitro formation of cloned human tissues by means of cell therapy. Several growth promoting agents are disclosed for use in a serum-free culture medium of normal human keratinocytes. Lithium ions, dibutryl-cyclic adenosine monophosphate, and prostaglandin E1 have been found effective as growth enhancing agents to be added singly or in combination, when used in combination with insulin-like growth factor-1. Lithium ions and prostaglandin E1 are disclosed as independent growth enhancing factors, that replace epidermal growth factor as a necessary growth factor required for keratinocyte clonal growth.

### CROSS REFERENCE TO RELATED APPLICATION

This application claims priority from provisional Application Ser. No. 61/351,312 filed Jun. 4, 2010, entitled “Enhancement of epidermal growth by non-protein growth factors”.

### BACKGROUND OF THE INVENTION

The serum-free culture of adult human epithelial stem cells is of paramount importance for the in vitro formation of cloned human tissues by means of cell therapy, and eventually for the success of regenerative medicine's approach to replace diseased, injured, “worn-out/aged” and lost host tissue. There were many early attempts to design a chemically defined media for the growth of epiderrmal keratinocytes (Peehl and Ham, 1980; Tsao M. C. et al., 1982; Boyce and Ham, 1983; Ham, R., 1984; Boyce and Ham, R., 1984; Nissley, P. et al., 1985; Boiseau A-M., et al., 1991). Earlier patent literature discloses serum-free media compositions to grow skin and other epithelial cells in culture (Boyce, S. T. and Ham, R., “Process and defined medium for growth of human keratinocyte cells”, U.S. Pat. No. 4,673,649 issued Jun. 16, 1987; Cheesebeuf M. L., et al., “Serum-free animal cell culture medium and methods for the primary culture and production of cell lines using this medium”, U.S. Pat. No. 4,786,599 issued Nov. 22, 1988; Chan S. Y., “Chemically defined growth medium”, U.S. Pat. No. 4,851,346 issued 1989; Boyce, S. T., and Ham, R., “Process and defined medium for growth of human epidermal keratinocyte cells”, U.S. Pat. No. 4,940,666 issued Jul. 10, 1990; Wolfe R. A., et al., “Basal nutrient medium for cell culture”, U.S. Pat. No. 5,232,848 issued Aug. 3, 1993; Boyce, S., “Method and apparatus for preparing composite skin replacement”, U.S. Pat. No. 5,273,900 issued Dec. 28, 1993; Wille, J. J., “Methods for the formation of a histologically complete skin substitute”, U.S. Pat. No. 5,292,655 issued Mar. 8, 1994; Keen M. J. and Rapson, N. T., “Defined media for serum-free tissue culture”, U.S. Pat. No. 5,316,938 issued May 31, 1994; Lindstrom, R. L., et al., “Method and apparatus defined serum-free medical solution”, U.S. Pat. No. 5,407,669 issued Apr. 18, 1995; Cole K. H. et al., “Human liver epithelial cell line and culture media therefor”, U.S. Pat. No. 5,529,920 issued Jun. 25, 1996; Wille, J. J., “Serum-free medium for use in the formation of a histologically complete living human skin substitute”, U.S. Pat. No. 5,686,307 issued Nov. 11, 1997; Wille, J. J., “Cell competency solution for use in the formation of a complete living human skin substitute”, U.S. Pat. No. 5,795,781 issued Aug. 18, 1998; Van Bossuyt H., “Non-viable total keratinocyte lysate for promoting wound healing”, U.S. Pat. No. 5,866,167 issued Feb. 2, 1999; Rees R., et al., “Wound treatment with keratinocytes on a solid support enclosed in a porous material”, U.S. Pat. No. 5,972,332; Dimoudis, N., et al., “Keratinocytes attached to microcarriers for treatment of skin wounds”, U.S. Pat. No. 5,980,888 issued Nov. 9, 1999; Wille, J. J., “Process and media for the growth of human epithelia”, U.S. Pat. No. 5,834,312 issued Nov. 10, 1998; Wille, J. J., “Process and media for the growth of human cornea and gingival”, U.S. Pat. No. 5,912,175 issued Jun. 15, 1999; Wille, J. J., “Hepes based nutrient medium for the isolation and culturing of stem cells”, U.S. Pat. No. 6,162,643 issued Dec. 19, 2000; Kuri-Harcuch, W. and Bolivar-Floresa, Y., “Methods of promoting healing of skin resurfacing wounds”, U.S. Pat. No. 6,713,084 issued Mar. 30, 2004.

An important advance in the serum-free culture of epidermal keratinocytes was the recognition that the requirement for protein growth such as epidermal growth factor (EGF) and insulin (Ins) can be replaced in a completely defined basal nutrient medium by insulin-like growth factor-1 (IGF-1) (Wille, J. J., U.S. Pat. No. 5,292,655 issued Mar. 8, 1994); and retinoids (Varani et al., 1989) and Wille, J. J., “Protein-free defined media for the growth of normal human keratinocytes”, U.S. Pat. No. 7,037,721 issued May 2, 2006), and Wille, J. J., “Skin healing compositions”, U.S. Provisional Patent No. 2009-0131537-A1. The latter patent discloses a composition that substitutes EGF for retinyl acetate at physioloigical concentrations for EGF, and eliminates the requirement for bovine pituitary extract (BPE), but the serum-free composition still requires insulin or IGF-1.

Trace metal ions are necessary for the long-term culture of keratinocytes and are included in most of the serum-free medium compositions cited above. Strontium ions (Sr2) are known to substitute for calcium in many other biological responses. Recently, the role of Strontium ions (Sr 2+), an unusual or exotic metal ion, was reported to enhance the growth of keratinocyte cells either in low calcium (0.03 mM) or in a calcium-free, serum-free medium (Praeger et al. 1987; Furukama et al. 1988). The latter author reported that the maximal enhancement of cell viability and increase in cell number occurred at between 1.0-2.0 mM. Lithium ions (Li+) can substitute for sodium ions in biological systems but because it has a smaller atomic radius, it forms stronger ionic bonds and can interfere with morphologoical movements and gene expression in the slime mold, dictyostelium discoideum (Peters et al. 1989) and embryological processes such as gastrulation in Sea Urchin (Nocente-McGrath et al. 1991), and amphibian embryos (Lazou and Beis, 1993). A possible mechanism by which lithium ions interfere with cellular movements is by stabilizing cytoskeleton F-actin fibers (Oloimbo et al. 1991; Dalle Donne et al. 1993). More relevant to effects on cell growth is a report that Li+ ions causes morphological alterations in various mammalian cell lines (Matthopoulis et al. 1996) at concentrations above 4 mM. The mechanism of action of lithium ions is largely unknown but it can affect the intracellular phosphoinositide signaling pathway through depletion of the intermediate, inositol triphosphate. In addition, when added to the culture medium of the ciliate, Blepharisma, it had an inhibitory effect of that cells photoresponses (Fabczak et al. 2005). Lithium ions at 15 mM when added to culture medium of the protozoan parasite, Herpetomonas, stimulated its growth (Nakamura and Pinto, 1989). Lithium ions at 20 mM concentration inhibited glucose synthesis in the rat liver (Bosch et al. 1992). Most relevant to the present invention is a report that lithium ions at a concentration of 2-20 mM had a stimulatory effect on insulin induced uptake of α-aminobutryic acid, synthesis of DNA and RNA and cell multiplication of mouse mammary gland explants cultured in a chemically-defined synthetic medium (Hari and Oka, 1979). The present invention discloses that lithium ions at 10 mM concentration stimulate the multiplication of human keratinocyte cells in a serum-free chemically defined medium as previously reported (Wille, 2008). The present invention also discloses that dibutryl-cyclic 3′-5′-cyclic adenosine monphosphate (diB-C-AMP) and prostaglandin E1 (PGD E1) are two other non-protein factors that enhance cell multiplication and clonal growth of normal human keratinocytes in a serum-free chemically defined culture medium, either singly or in combination with lithium ions.

Facts concerning the structure, synthesis, decomposition and biological functionality of cAMP can be accessed on-line at: http://en.wikipedia,org/wiki/cAMP. It is synthesized from ATP by adenyl cyclase, a plasma membrane enzyme, which is ordinarily activated by hormone ligand occupation of specific cell surface hormone receptors like the ligand insulin. cAMP is an intracellular second messenger that is involved in a signaling cascade, which acts through a cAMP-dependent protein kinase PKA) that phosphorylates nuclear DNA binding proteins, thereby turning on select DNA sequences for gene expression. cAMP also activates calcium channels, providing a minor pathway by which growth hormone releasing hormone causes a release of growth hormone. In non-human systems, cAMP is a chemoattractant for slime mold cells.

Like lithium ions, cAMP through cAMP-dependent PKA regulates actin organization and cell motility (Glenn, 2003). cAMP does not readily cross the plasma membrane of mammalian cells. Therefore, when one studies the action of cAMP on cells in culture, the more membrane-soluble derivative, dibutryl-cyclic AMP (diBCAMP) is usually employed. An early study reported that diBCAMP inhibited epidermal cell division (Voorhees et al. 1972a), and also reported that decreased concentration of cAMP in the epidermis of psoriatic lesions (Voorhees et al, 1972b). These results appear to contradict later reports that addition of cholera toxin, a potent toxin, that induces an increase in intracellular cAMP, and actually enhances keratinocyte growth (Green, 1978). Finally, addition of chlora toxin had no effect of human keratinocyte growth in a fetal serum-supplemented medium (Pheel and Ham, 1980). These disparate results may also reflect complex interactions and unpredictable consequences attributable to employing serum-containing culture media.

Pursuant to the present invention, it has been found that diBCAMP does enhance the clonal growth of normal human keratinocytes in serum-free, chemically defined media.

An aim of the present invention is to discover the effect of pro-inflammatory mediators on the growth of normal human keratinocytes. The role of ecosinoids as proinflammatory mediators of the arachidonic acid casacade is well-documented (Ikai, 2000). Less is known about their effects on proliferation of epidermal keratinoytes, and even less about their effects in controlled tissue culture studies. Human skin can generate eicosanoids, which were reported to be involved in the regulation of growth and differentiation of the epithelia (Ikai, 2000). Actually, prostaglandins probably do not play a central role in inflammatory skin diseases. Prostaglandin E1 (PGE1) is a drug used in the treatment of erectile dysfunction (see structure and physiological attributes on line at http://en.wikipedia.org.wiki/Prostaglandin E1). PGE1 is a potent vasodilator and antithrombotic and injection of it have been widely used in circulatory disturbances in skin ulcers and various collagen diseases. PGE1 has also shown clinical effectiveness in an ointment for the treatment of burn wounds (Gunji et al. 1996).

Yet another aim of the present invention is to discover the effect of prostaglandins in cell multiplication. PGE1 stimulates chloride secretion in a colonic epithelial cell line, which is associated with an increase in cyclic AMP level (Weymer et al. 1985). In addition, PGE1 and lithium have been reported to enhance the protective effects of heat shock proteins of neurons against ischemic injury (Han et al. 2008). PGE1 is reported to increase EGF production in three-dimensional cultured human annulus cells (Gruber et al. 2009). A combination of ovine prolactin (0.1 microgram/ml) and prostaglandin E1 (2.5×10−8M) supports clonal growth of early passage human mammary epithelial cells in a serum-free, chemically defined, synthetic culture medium (Hammond et al. 1984). PGE1 has also been included in selective media for keratinocytes and fibroblasts (Ham, 1984). For human dermal fibroblasts PGE1 is only of slightly beneficial value and substantial serum-free growth occurs without it (Bettger et al. 1981).

A prior patent disclosure reported a protein-free serum version of a free medium that was designed to support the serial propagation of normal human keratinocytes (NHK). It accomplished this by substituting a physiological concentration of retinyl acetate for the protein growth factors insulin (Ins) and epidermal growth factor (EGF) (Wille, J. J. “Protein-free defined medium for the growth of normal human keratinocytes”, U.S. Pat. No. 7,037,721 issued May 2, 2006).

Another aim of the present invention is to employ the chemically defined basal nutrient medium realized in the above “protein free-medium” composition to examine the effectiveness of the novel non-protein growth enhancing agents—lithium ions, diBcAMP, and PGE1—using the technique of clonal growth assays on cultures of normal human keratinocytes.

### SUMMARY OF THE INVENTION

The primary aim of the present invention is to provide a superior chemically defined, serum-free culture medium. For this purpose, the present invention builds on the teaching of a prior patent (Wille, J J., U.S. Pat. No. 7,037,731-2006), which discloses a novel serum-free medium composition and a second patent (Wille J. J., U.S. Pat. No. 5,696,307-1997), which discloses the advantage of a protein-free, chemically defined, serum-free medium composition that uses a retinoid to replace epidermal growth factor (EGF). This was shown to achieve high performance culture of normal human keratinocytes. The present invention embodies the previous Wille patent disclosures as necessary elements in defining the activities of several new non-protein growth factors that enhance the growth and cell multiplication of normal human keratinocytes in combination with the above-noted chemically defined, serum-free serum medium, here designated HECK 109. In particular, clonal growth assays were employed to determine the effects of supplementing a standardized serum-free medium composition either individually or in combination with the following three non-protein growth factor additives: (1) lithium ions (Li+), (2) a cyclic adenosine monophosphate derivative, dibutryl-3′-5′-adenosine monophosphate (diBcAMP), and (3) prostaglandin E 1 (PGE1). The effects of these additives were examined under in the presence of low (0.1 mM) concentration of calcium ions in the medium. In addition, clonal growth assays were employed to examine the modifying effect of varying concentrations of six key amino acids, i.e., low (1×) versus high concentrations (3×) of histidine, isoleucine, methionine, phenylalanine, tryptophane, and tyrosine. The concentrations of these are given in Table 1.

In summary, the results of the clonal growth assays revealed both independent growth stimulation of each of the non-protein factors and synergistic stimulation by pair-wise combinations of these non-protein growth factors in combination with two protein growth factors, insulin-like growth factor-1 (IGF-1) alone or IGF-1 plus epidermal growth factor (EGF), as exemplified in the body of the specification by the Examples below.

### DESCRIPTION OF THE DRAWINGS

FIG. 1: Photographs showing the enhancing effect of diButryl cyclic adenosine monophosphate (diBcAMP) and lithium ions (Li+) on clonal growth of normal human keratinocytes. A) Control clonal growth assay dishes: (top left), labeled IGF-1+EGF, d1 (day one) and (top right), labeled IGF-1+EGF, d10 (day ten). B) Experimental clonal growth assay dishes: culture medium containing IGF-1 (5 ng/mL) supplemented with diBcAMP (0.1 mM) (bottom left), labeled IGF-1+diBcAMP; culture medium supplemented with IGF-1 (0.1 mM) and Li+ ions (bottom right). All dishes were fixed with glutaraldehyde and stained with 0.2% Crystal Violet stain. The dishes were photographed (1×, magnification).

FIG. 2: Photographs showing the enhancing effect of prostaglandin E1 (PGE1) in combination with IGF-1 or insulin (Ins) in the presence or absence of epidermal growth factor (EGF) on the clonal growth of normal human keratincytes. A) Control culture dishes: (top left), labeled EGF+IGF-1, (d1, day one); (middle bottom), labeled EGF+IGF-1 (d10, day 10). B) Experimental culture dishes: (top middle), labeled EGF+IGF-1+PGE1, d10 (day ten); (top right), labeled IGF-1+PGE1 (d10); bottom right-labeled PGE1 (d10). All dishes were fixed with 5% glutaraldehyde and stained with 0.2% Crystal Violet stain. The dishes were photographed (1×, magnification).

FIG. 3: Photographs of clonal growth assay culture dishes showing the enhancing effect of PGE1 in combination with EGF and Insulin cultured in the presence of 1× level of amino acid (left panel) and in presence of 3× amino acids (right panel) on the clonal growth of normal human keratincytes. A) Control culture dishes supplemented with I+/E+; B) experimental supplemented with I+/E+/PGE1. All dishes were fixed with 5% glutaraldehyde and stained with 0.2% Crystal Violet stain. The dishes were photographed (1×, magnification).

FIG. 4: Photographs of clonal growth assay culture dishes showing the enhancing effect of lithium ions (Li+) in combination with EGF and IGF-1 when cultured in the presence of 1× amino acid (left panel), and no enhancement when cultured in presence of 3× amino acids (right panel) on the clonal growth of normal human keratincytes. A) Control culture dishes supplemented with insulin (I+) and EGF (E+) only; B) experimental culture dishes supplemented with I+ and E+plus PGE1. All dishes were fixed with 5% glutaraldehyde and stained with 0.2% Crystal Violet stain. The dishes were photographed (1×, magnification).

### DETAILED DESCRIPTION OF THE PREFERRED EMBODIMENTS

### Example 1

### Methods for Clonal Growth Assays

The methods for isolating and culturing primary and secondary-passaged normal human neonatal normal human keratinocyte (NHK cells) were those previously employed (Wille et al., 1984). For the purpose of the present invention, secondary serially-passaged cultures were plated at 500 cells/cm in a novel serum-free basal media previously disclosed (Wille J. J., U.S. Pat. No. 5,586,307). This basal nutrient defined culture medium was supplemented either singly with the following additives: diBCAMP (1×10−4 M), LiCl (10 mM), and PGE1 (10 μg/ml) in the above media supplemented with Insulin-like growth factor (IGF-1, 5 ng/mL) and epidermal growth factor (EGF, 5-10 ng/mL). All dishes were fixed ten days later, stained with crystal violet stain (0.2%) and photographed.

### Example 2

### Growth Stimulation of NHK by Single Addition of DiBCAMP and Li+, Non-Protein Growth Factors

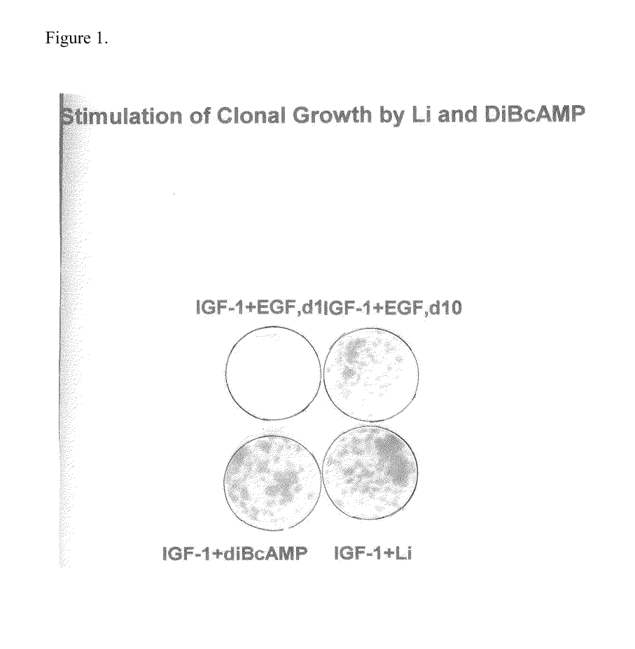

FIG. 1 presents photographs showing the results of several clonal growth assays. It shows that 10 days after plating cells, the clonal growth of NHK supplemented with both IGF-1 and diBcAMP is significantly enhanced relative to a control medium supplemented with IGF-1 and EGF. Further, it definitively shows that DiBcAMP can replace EGF, which ordinarily is a necessary growth factor requirement for the clonal growth of NHK cells. FIG. 1 also presents the results of clonal growth assays showing that Li+ ions in combination with IGF-1 significantly enhances clonal growth of a NHK relative culture medium containing EGF plus IGF-1. This result demonstrates that Li+ ions can replace EGF as a necessary requirement for clonal growth. The importance of these results lies in the benefit of replacing EGF with low cost non-protein growth factors.

### Example 3

### Enhancement of Clonal Growth of NHK by Single Addition of Prostaglandin E1

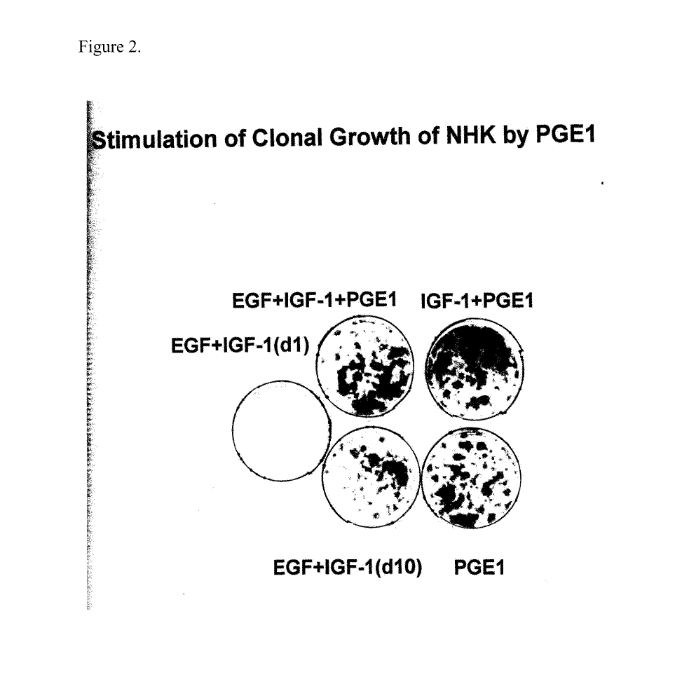

FIG. 2 presents photographs showing that after 10 days of clonal growth, the single addition of PGE1 to culture medium 1) unsupplemented by any protein growth factors (bottom right), supplemented with a combination of PGE1 and IGF-1 (top right), and culture medium supplemented with PGE1 in combination with both EGF and IGF-1 (top left) significantly enhanced the clonal growth of the NHK relative culture medium supplemented with both only EGF plus IGF-1. This result demonstrates that PGE1 is a potent growth factor that can replace both EGF and IGF-1, two protein growth factors that are absolutely required for clonal growth of NHK.

### Example 4

### Effect of Elevated Levels of Key Amino Acid on PGE1 and Li+ Ion-Induced Enhancement of Clonal Growth

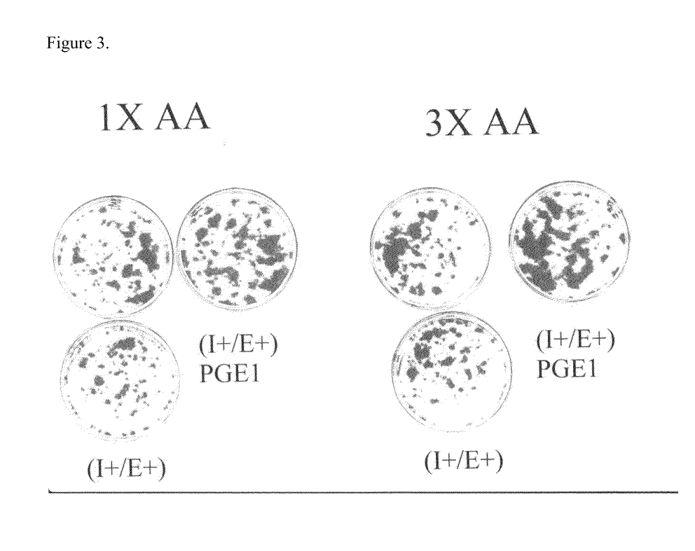

A second study was undertaken to examine the effect of lithium ions, and PGE1 in a serum-free basal medium to which the following six amino acids (his, met, phe, tryt, and tyr) were increased by three times (3×) their normal concentration. FIG. 3 shows that mere elevation of the six key amino acids (histidine, isoleucine methionine, phenylalanine, tryptophane and tyrosine) had no positive stimulatory effect on clonal growth in a control medium supplemented with the two protein growth factors: Insulin (I+, 5 μg/mL) and EGF (E+, 10 ng.mL) relative to 1× level of the amino acid. By contrast, FIG. 3 reveals a significant enhancement of clonal growth in a culture medium supplemented with PGE1 in elevated levels of the six key amino acids relative to the lower (1×) levels of amino acid when cultured in the presence of I+ and E+ protein growth factors. This result indicates that the strength of the stimulatory effect of PGE1 is dependent on the nutritional action of elevated levels of amino acids.

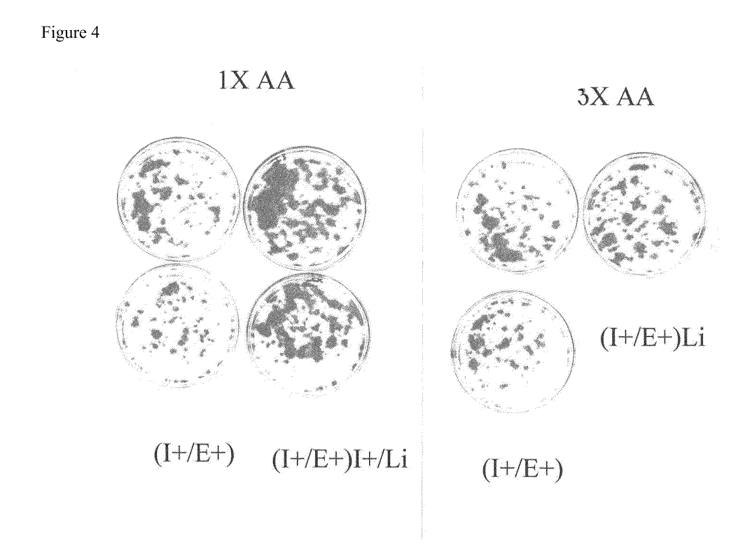

FIG. 4 present photographs of clonal growth assays showing that the growth-enhancing effects of lithium ions, seen in low (1×) levels of the six key amino acid is lost in high levels (3×) of the key amino acids. This may mean that the Li+ion is sequestered in the medium containing high levels of amino acid.

### Example 5

### Composition of HECK 109 Chemically Defined Basal Nutrient Medium Severally Supplemented with One of the Three Non-Protein Growth Factors

Enhancement of keratinocyte clonal growth may be achieved by supplementing many existing basal nutrient media that are commonly employed as the chemically defined medium suitable for serum-free culture of normal human keratinocytes. In this example we choose HECK 109 basal nutrient medium.

TABLE 1
HECK 109: Basal Nutrient Medium Composition Supplemented by
Non-Protein Growth Factor Actives.

| | | Concentration in final medium | |
|---|---|---|---|
| Stock | Component | mg/l | mol/l* |
| 1 | Arginine•HCl | 210.7 | 1.00 × 10−3 |
| | Histidine•HCl•H20 | 33.54 | 1.60 × 10−4 |
| | Isoleucine allo-free | 6.6 | 4.50 × 10−5 |
| | Leucine | 66.0 | 0.50 × 10−3 |
| | Lysine•HCl | 18.3 | 1.00 × 10−4 |
| | Methionine | 8.95 | 6.00 × 10−5 |
| | Phenylalanine | 16.67 | 1.00 × 10−4 |
| | Threonine | 23.8 | 2.00 × 10−4 |
| | Tryptophan | 10.2 | 0.50 × 10−4 |
| | Tyrosine | 5.40 | 3.00 × 10−5 |
| | Valine | 35.13 | 3.00 × 10−4 |
| | Choline | 13.96 | 1.00 × 10−4 |
| | Serine | 63.06 | 6.00 × 10−4 |
| 2 | Biotin | 0.0146 | 6.00 × 10−8 |
| | Calcium Pantothenate | 0.285 | 1.00 × 10−6 |
| | Niacinamide | 0.03663 | 3.00 × 10−7 |
| | Pyridoxal•HCl | 0.06171 | 3.00 × 10−7 |
| | Thiamine•HCl | 0.3373 | 1.00 × 10−6 |
| | Potassium chloride | 111.83 | 1.50 × 10−3 |
| 3 | Folic acid | 0.79 | 1.80 × 10−6 |
| | Na2HPO4•7H20 | 536.2 | 2.00 × 10−3 |
| 4a | Calcium chloride•2H20 | 14.7 | 1.00 × 10−4 |
| 4b | Magnesium chloride•6H20 | 122.0 | 6.00 × 10−4 |
| 4c | Ferrous sulfate•7H20 | 1.39 | 5.00 × 10−6 |
| 5 | Phenol red | 1.242 | 3.30 × 10−6 |
| 6a | Glutamine | 877.2 | 6.00 × 10−3 |
| 6b | Sodium pyruvate | 55.0 | 5.00 × 10−4 |
| 6c | Riboflavin | 0.03764 | 1.00 × 10−7 |
| 7 | Cysteine•HCl | 37.6 | 2.40 × 10−4 |
| 8 | Asparagine | 13.2 | 1.00 × 10−4 |
| | Proline | 34.53 | 3.00 × 10−4 |
| | Putrescine | 0.1611 | 1.00 × 10−6 |
| | Vitamin B12 | 0.407 | 3.00 × 10−7 |
| 9 | Alanine | 8.91 | 1.00 × 10−4 |
| | Aspartic acid | 3.99 | 3.00 × 10−5 |
| | Glutamic acid | 14.71 | 1.00 × 10−4 |
| | Glycine | 7.51 | 1.00 × 10−4 |
| 10 | Adenine | 12.16 | 9.00 × 10−5 |
| | Inositol | 18.02 | 1.00 × 10−4 |
| | Lipoic acid | 0.2063 | 1.00 × 10−6 |
| | Thymidine | 0.7266 | 3.00 × 10−6 |
| | Trace Elements | | |
| | Copper sulfate•5H20 | 0.00025 | 1.00 × 10−9 |
| | Selenic acid | 0.00387 | 3.00 × 10−8 |
| | Manganese sulfate•5H20 | 0.00015 | 1.00 × 10−9 |
| | Sodium silicate•9H20 | 0.1421 | 5.00 × 10−7 |
| | Ammonium molybdate•4H20 | 0.00124 | 1.00 × 10−9 |
| | Ammonium vanadate | 0.00059 | 5.00 × 10−9 |
| | Nickel chloride•6H20 | 0.00012 | 5.00 × 10−10 |
| | Stannous chloride•2H20 | 0.000113 | 5.00 × 10−10 |
| | Zinc chloride•7H20 | 0.1438 | 5.00 × 10−7 |
| | Solids | | |
| | Glucose | 1081.0 | 6.00 × 10−3 |
| | Sodium acetate•3H20 | 500.0 | 3.70 × 10−3 |
| | Sodium bicarbonate | 1176.0 | 1.40 × 10−2 |
| | Sodium chloride | 7022.0 | 1.20 × 10−2 |
| | HEPES | 5240.0 | 2.20 × 10−2 |
| | Non-protein actives | | |
| | Lithium chloride | | 1 × 10−2 |
| | Dibutryl-3′-5′-cyclic AMP | | 1 × 10−4 |
| | Prostaglandin E1 | | 2.82 × 10−2 |
| | Hormones | | |
| | Ethanolamine | 6.1 | 1 × 10−4 |
| | Phosphoethanolamine | 14.11 | 1 × 10−4 |
| | Hydrocortisone | 0.0363 | 5 × 10−7 |

The above medium formulation must be supplemented with at least one of the listed non-protein growth factors and lithium and PGE1 require IGF-1, whereas dibutryl C-AMP requires both IGF-1 and EGF as protein growth factors.

There have thus been shown and described three different non-protein growth enhancing factors. Each is an additive that can be added to a chemically defined serum-free culture medium suitable for the growth and proliferation of rapidly growing normal human keratinocytes. Lithium ions presented as a salt, lithium chloride, is readily soluble in the aqueous phase of the medium and can be added aspetically as a 1:100 fold dilution and as the terminal component from a 1M LiCl concentrated stock solution to give a final concentration of 10 mM. Likewise, a 100 mM stock solution of dibutryl-cAMP dissolved in alcohol can be diluted 1:1000 aseptically to yield a final concentration of 0.1 mM and added as the terminal component to the final basal nutrient medium. Finally, 1 mg/mL stock solution of prostaglandin E1 can be diluted 1:1000 aseptically to yield a final concentration of 10 μg/mL in the culture medium as the terminal component of the medium.

Detailed and extensive in vitro tissue culture studies showed that each of non-protein growth factors significantly increased the cell number and size of the keratinocyte colonies. Lithium ions completely replaced EGF as a necessary protein growth factor for clonal growth of keratinocytes. Thereby, lithium ions qualify per se as an independent growth factor. It is to be understood, that the examples produced of medium composition that can be improved by addition of one or more, and combinations of, the named non-protein growth factors are not limited to those alone but can be any of the many commercially available serum-free culture media designed for keratinocytes and other epithelial cell types. It is anticipated that the use of these non-protein growth factor supplements and derivative formulations containing these factors may be adopted as allied applications that are available to one familiar with the state of the art in formulating culture media and compositions. Many changes, modifications, variations and other uses and applications of the subject invention will, however, become apparent to those skilled in the art after considering the specification and the accompanying changes, modifications, variations and other uses and applications which do not depart from the spirit and scope of the invention are deemed to be covered by the invention, which is not to be limited only by the claims which follow.

What is claimed is:

1. A method of growing human keratinocytes for use in producing cloned human tissue, said method comprising the steps of:
(a) providing a serum-free culture as a basal nutrient medium for cell growth;
(b) adding epithelial cells from a human body to said medium;
(c) adding at least one non-protein growth factor to said medium to enhance the growth of normal human keratinocytes;
wherein said non-protein growth factor is selected from the group consisting of lithium ions, prostaglandin E1 and dibutryl-cyclic adenosine monophosphate.

2. The method defined in claim 1, wherein the medium is chemically defined.

3. The method defined in claim 2, wherein the composition of said chemically defined medium is adjusted for optimal growth of keratinocytes.

4. The method defined in claim 2, wherein the chemically defined medium is HECK 109.

5. The method defined in claim 1, wherein the non-protein growth factors include lithium ions used in the concentration range of 0.1 to 10 millimoles.

6. The method defined in claim 1, wherein the non-protein growth factors include prostaglandin E1 used in the concentration range of 0.1 to 10 micrograms per milliliter.

7. The method defined in claim 1, wherein the non-protein growth factors include dibutryl-cyclic adenosine monophosphate used in the concentration range of 1×10−6 M to 1×10−4 M.

8. The method defined in claim 1, wherein two of said non-protein growth factors are added to said medium.

9. The method defined in claim 1, wherein all three of said non-protein growth factors are added to said medium.
